(19) United States
(12) Patent Application Publication
(10) Pub. No.: US 2025/0143875 A1
(43) Pub. Date: May 8, 2025

(54) PROSTHETIC HEART VALVE

(71) Applicant: EDWARDS LIFESCIENCES CORPORATION, Irvine, CA (US)

(72) Inventors: Tamir S. Levi, Zikhron Yaakov (IL); Elena Sherman, Pardes Hana (IL)

(21) Appl. No.: 19/015,448

(22) Filed: Jan. 9, 2025

Related U.S. Application Data
(63) Continuation of application No. PCT/US2023/027330, filed on Jul. 11, 2023.
(60) Provisional application No. 63/368,238, filed on Jul. 12, 2022.

(51) Int. Cl. A61F 2/24

(52) U.S. Cl. CPC A61F 2/2418; A61F 2210/0057

(57) ABSTRACT

A prosthetic valve can include an annular frame that is radially expandable and compressible, a skirt member attached to the annular frame, and a protective member attached to an inner surface the skirt member. The protective member is less abrasive than the skirt member and extends circumferentially around at least a portion of the annular frame.

### CROSS-REFERENCE TO RELATED APPLICATIONS

This application is a continuation of PCT Patent Application No. PCT/US2023/027330 filed on Jul. 11, 2023, which claims the benefit of U.S. Provisional Application No. 63/368,238, filed Jul. 12, 2022, each of these applications being incorporated by reference herein in its entirety.

### FIELD

The present disclosure relates to implantable, radially expandable prosthetic devices, such as prosthetic heart valves, and to methods, assemblies, and apparatuses for delivering, expanding, implanting, and deploying such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve (or simply “prosthetic valve”) can be mounted in a crimped state on the distal end of a delivery apparatus and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

The prosthetic valve can include a radially compressible and expandable frame and a leaflet structure mounted within the frame. In some circumstances, the prosthetic valve can have a sealing member, such as an outer skirt mounted on an outer surface of the frame. The outer skirt can be configured to seal against surrounding native tissue so as to reduce paravalvular leakage past the prosthetic valve.

Despite the recent advancements in percutaneous valve technology, there remains a need for improved transcatheter prosthetic valves.

### SUMMARY

The present disclosure relates to methods and devices for treating valvular diseases. Specifically, the present disclosure is directed to implantable, radially expandable prosthetic devices, such as prosthetic heart valves, and to methods, assemblies, and apparatuses for delivering, expanding, implanting, and deploying such prosthetic devices.

According to certain aspects of the disclosure, a prosthetic valve can include an annular frame that is radially expandable and compressible, a skirt member attached to the annular frame, and a protective member attached to an inner surface the skirt member. The protective member is less abrasive to leaflets of the prosthetic valve than the skirt member and extends circumferentially around at least a portion of the annular frame.

According to certain aspects of the disclosure, a prosthetic valve can include an annular frame that is radially expandable and compressible, and a skirt assembly attached to the annular frame. The skirt assembly can include a skirt member and a protective member attached to an inner surface of the skirt member. The protective member can be located at an outflow end portion of the skirt member and extends circumferentially around at least a portion of the annular frame.

According to certain aspects of the disclosure, a prosthetic valve can include an annular frame that is radially expandable and compressible, and a skirt member attached to the annular frame. The skirt member can include a body portion and an outflow end portion. The outflow end portion is less abrasive than the body portion.

According to certain aspects of the disclosure, a prosthetic valve can include an annular frame that is radially expandable and compressible, a plurality of leaflets mounted inside the annular frame, a skirt member attached to the annular frame, and a protective member attached to an inner surface the skirt member. The plurality of leaflets can be movable between an open configuration and a closed configuration. In the open configuration, the plurality of leaflets can, in some instances, contact the protective member.

According to certain aspects of the disclosure, a prosthetic valve can include an annular frame that is radially expandable and compressible, a skirt member attached to the annular frame, and a protective member attached to an inner surface the skirt member. The protective member can be configured to shield a terminal end of the skirt member from contacting one or more leaflets mounted within the annular frame.

Certain aspects of the disclosure also concern a skirt assembly for a prosthetic valve. The skirt assembly can include a skirt member having an annular shape and a protective member attached to an outflow end portion of the skirt member. The protective member can extend circumferentially around an inner surface of the skirt member. The protective member is less abrasive to leaflets of the prosthetic valve than the skirt member.

Certain aspects of the disclosure also concern an assembly including any one of the prosthetic valves described above, and a delivery apparatus configured to deliver the prosthetic valve in the radially compressed state to a target location.

Certain aspects of the disclosure also concern a method of assembling a prosthetic valve. The method can include preparing a skirt member having an annular shape and affixing a protective member to an outflow end portion of the skirt member. The protective member encircles an inner surface of the skirt member. The protective member is less abrasive to leaflets of the prosthetic valve than the skirt member.

Certain aspects of the disclosure concern a method including delivering a prosthetic device in a radially compressed state to a target location, and radially expanding the prosthetic device to a radially expanded state. The prosthetic device can be any one of the prosthetic valves described above.

The above method can be performed on a living animal or on a simulation, such as on a cadaver, cadaver heart, anthropomorphic ghost, simulator (for example, with body parts, heart, tissue, etc. being simulated).

In some examples, a prosthetic valve comprises one or more of the components recited in Examples 1-108 described in the section “Additional Examples of the Disclosed Technology” below.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a perspective view of a prosthetic valve, according to one example.

FIG. 2 is a perspective view of a frame of the prosthetic valve of FIG. 1.

FIG. 3 is a side view of a delivery apparatus configured to deliver and implant a prosthetic valve at a target implantation site, according to one example.

FIG. 4A is a front view of a portion of a flattened outer skirt assembly, according to one example.

FIG. 4B is a back view of the portion of the flattened outer skirt assembly of FIG. 4A.

FIG. 4C is a cross-sectional view of an outer skirt assembly and a leaflet taken along line 4C-4C depicted in FIG. 1, according to one example.

FIG. 5A is a side view of a straightened protective member which is twisted along its longitudinal axis, according to one example.

FIG. 5B is a side view of a straightened protective member which is twisted along its longitudinal axis and then shape-set to have a flat configuration.

FIG. 6 depicts a portion of the prosthetic valve of FIG. 1 viewed from an interior of the prosthetic valve, according to one example.

FIG. 7 depicts a portion of the prosthetic valve of FIG. 1 viewed from an interior of the prosthetic valve, according to another example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. Further, as used herein, “and/or” means “and” or “or,” as well as “and” and “or.”

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

Directions and other relative references (for example, inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

### General Overview of Prosthetic Valves

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

According to certain examples, any of the prosthetic valves disclosed herein can be adapted to be implanted in the native aortic annulus. In other examples, any of the prosthetic valves disclosed herein can be adapted to be implanted in other native annuluses of the heart (for example, the pulmonary, mitral, and/or tricuspid valves). In certain examples, the disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries, and vessels of a patient. Additionally, the disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

### Exemplary Components of Prosthetic Valves

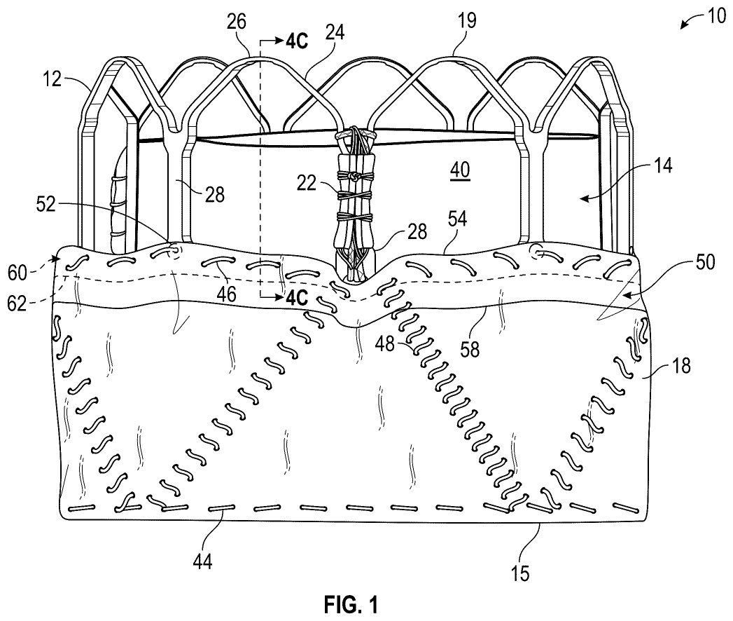

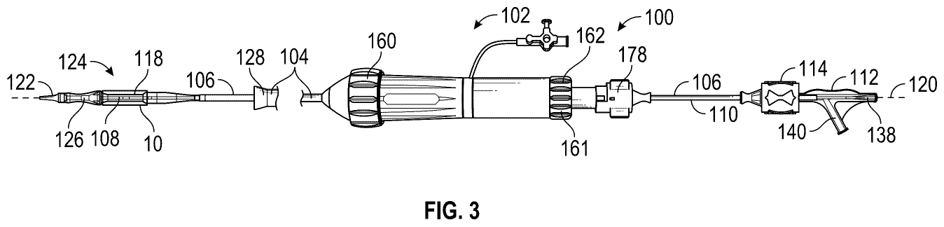

FIG. 1 illustrates an exemplary prosthetic valve 10 that can be advanced through a patient's vasculature, such as to a native heart valve, by a delivery apparatus, such as the exemplary delivery apparatus shown in FIG. 3.

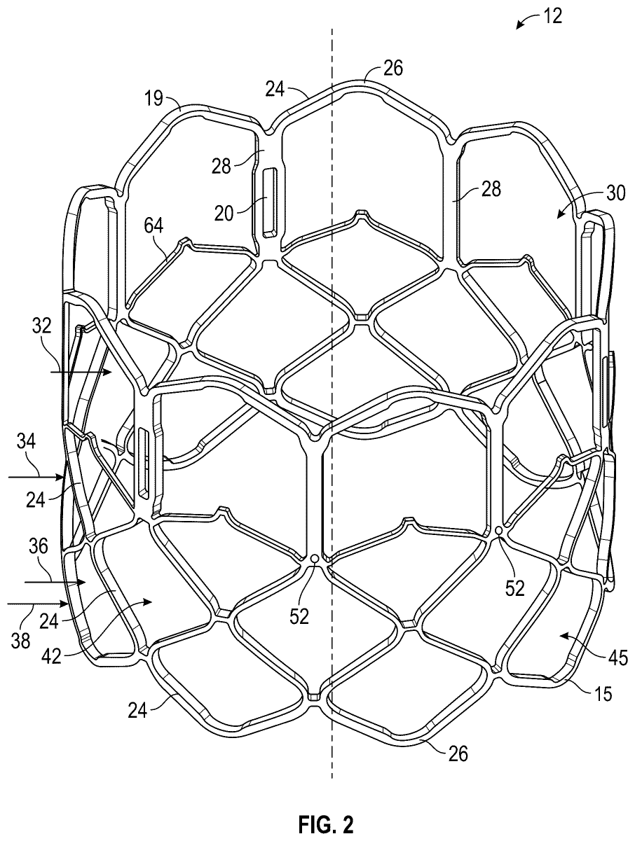

The prosthetic valve 10 can comprise a stent or frame 12, a valvular structure 14, and a perivalvular outer sealing member or outer skirt 18. An example frame 12 is depicted in FIG. 2.

The frame 12 can have an inflow end 15 and an outflow end 19. The valvular structure 14 can be configured to allow unidirectional flow of fluid (e.g., blood) through the prosthetic valve 10. In certain examples, the valvular structure 14 can include a plurality of leaflets 40, collectively forming a leaflet structure. The plurality of leaflets 40 can move between an open configuration and a closed configuration. The prosthetic valve 10 is configured for implantation in a native aortic annulus. During systole of the left ventricle, the leaflet structure is configured to permit blood flow from the inflow end 15 to the outflow end 19 (for example, the leaflets are in the open configuration). During diastole of the left ventricle, the leaflet structure is configured to block blood fluid flow from the outflow end 19 to the inflow end 15 (for example, the leaflets are in the closed configuration).

In certain examples, the valvular structure 14 can comprise three leaflets 40, which can be arranged to collapse in a tricuspid arrangement. In other examples, there can be greater or fewer number of leaflets (for example, one leaflet, two leaflets, or more than three leaflets).

The leaflets 40 can be secured to one another at their adjacent sides to form commissures 22 (or “commissure tabs”) of the leaflet structure. The lower (or inflow) edge of valvular structure 14 can define an undulating, curved scalloped shape, and thus can be referred to as a “scallop line.” In some examples, the leaflets 40 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

Additional examples of leaflet assemblies and methods for attaching leaflets to a frame are described in Provisional U.S. Patent Application No. 63/300,302, filed Jan. 18, 2022, and U.S. Patent Application No. 63/278,922, filed Nov. 12, 2021, which are incorporated by reference herein.

The frame 12 can be radially compressible (collapsible) and expandable (for example, the radially expanded configuration is shown in FIG. 1). The frame 12 can comprise a plurality of interconnected angled struts 24. A plurality of apices 26 that are spaced circumferentially apart can be formed at the inflow end 15 and the outflow end 19 of the frame 12. In FIG, 1, only the apices 26 at the outflow end 19 (also referred to as outflow apices) are visible, whereas the apices 26 at the inflow end 15 (also referred to as inflow apices) are covered by the outer skirt 18. Each apex 26 can be formed at a junction between two angled struts 24 at either the inflow end 15 or the outflow end 19.

The angled struts 24 can be arranged in multiple rows between the inflow end 15 and the outflow end 19. For example, FIG. 2 shows four rows of angled struts 24: a first row 32 of angled struts defining the outflow end 19, a second row 34 of angled struts upstream of the first row 32 of angled struts, a third row 36 of angled struts upstream of the second row 34 of angled struts, and a fourth row 38 of angled struts defining the inflow end 15. The first row 32 of angled struts can also be referred to as outflow struts, and the fourth row 38 of angled struts can also be referred to as inflow struts. In other examples, the frame 12 can include fewer (for example, 3) or more (for example, 5, 6, or more) rows of angled struts.

As described herein, a valve component (for example, a row of angled struts) is deemed to be upstream of a reference object (for example, another row of angled struts) if the valve component is closer to the inflow end 15 (or farther away from the outflow end 19) than the reference object.

The frame 12 can be formed with a plurality of circumferentially spaced commissure windows 20 that are adapted to mount the commissures 22 of the valvular structure 14 to the frame 12. For example, the frame 12 can include a plurality of axial frame members 28 bridging two adjacent rows (for example, the first row 32 and second row 34 of angled struts that are closest to the outflow end 19). The commissure windows 20 can be formed as through-thickness openings (for example, axially extending slots) on selected axial frame members 28. The commissures 22 can be inserted through the commissure windows 20 and secured to the selected axial frame members 28. Thus, the selected axial frame members 28 having the commissure windows 20 can also be referred to as commissure supports.

In other instances, the commissures of the leaflets can be secured to the frame in various other manners. For example, in some instances, the frame can be formed without commissure windows, and the commissures can, for example, be secured to the frame using sutures, fabric, and/or other means for coupling. Additional information for coupling the commissures to the frame, as well as other frame configurations, can be found, for example, in PCT Application No. PCT/US2022/012873 and U.S. Application No. [Attorney Docket No. 12783US01], both of which are incorporated by reference herein.

The axial frame members 28, together with the first row 32 of angled struts and the second row 34 of angled struts, can form a circumferentially extending row of open outflow cells 30. The first row 32 of angled struts can form the upper or outflow edges of the outflow cells, and the second row 34 of angled struts can form the lower or inflow edges of the outflow cells 30. The rows of angled struts upstream of the first row 32 (for example, rows 34, 36 and 38) can be interconnected so as to form additional rows of open cells. For example, FIG. 2 shows a second row of intermediate cells 42 disposed proximate an intermediate portion of the frame 12 and a third row of inflow cells 45 disposed at the inflow end 15 of the frame 12. The outflow cells 30 can have a hexagonal shape when the frame 12 is radially expanded. In certain examples, the outflow cells 30 can be more axially elongated and have a larger open area than the intermediate cells 42 and the inflow cells 45.

The outer skirt 18 can be arranged on and/or coupled to an outer surface of the frame 12. The outer skirt 18 (and any other outer skirts described herein) can be formed from any of various suitable biocompatible materials, including any of various synthetic materials, including fabrics (for example, polyethylene terephthalate fabric) or natural tissue (for example, pericardial tissue). The outer skirt 18 can function as a sealing member for the prosthetic valve 10 by sealing against the tissue of the native valve annulus and helping to reduce paravalvular leakage through and/or around the prosthetic valve 10.

The outer skirt 18 can extend from the inflow end 15 toward the outflow end 19 of the frame 12. In some examples, the outer skirt 18 can be secured to the struts of the frame 12 at the inflow end 15 by one or more sutures 44. In some examples, an outflow end portion 50 of the outer skirt 18 can be secured to the lower ends of the axial frame members 28 and/or to upper ends of the second row 34 of angled struts by one or more sutures 46. In some examples, a portion of the sutures 46 can extend through and be secured to apertures 52 located at the lower ends of the axial frame members 28 (apertures 52 shown in dashed circles in FIG. 1 to denote their position underneath the outer skirt 18).

In some examples, the sutures 46 and/or 44 can be in-and-out stitches. In some examples, the sutures 46 can be continuous between adjacent apertures 52 (for example, extending in and out of the outer skirt 18). In other examples, the sutures 46 can be separated pieces that individually connect the outflow end portion 50 of the outer skirt 18 to respective axial frame members 28.

In some examples, additional sutures 48, which can be configured as whip stitches, can further secure the outer skirt 18 to angled struts of the frame 12 disposed and extending between the inflow end 15 and the lower ends of selected axial frame members 28 (for example, the axial frame members 28 having commissure windows 20).

In some examples, the outflow end portion 50 of the outer skirt 18 can have about the same height (for example, having substantially constant axial distance relative to the inflow end 15) around the circumference of the frame 12. As such, the outer skirt 18 can cover at least a lower portion of the outflow cells 30, and an outflow edge 54 of the outer skirt 18 can extend between adjacent axial frame members 28.

In certain examples, a protective member 60 can be disposed on an inner surface of the outer skirt 18. The protective member can be affixed to the outer skirt 18 in various ways (for example, by the sutures 46). In FIG. 1, a lower end of the protective member 60 is marked by a dashed line.

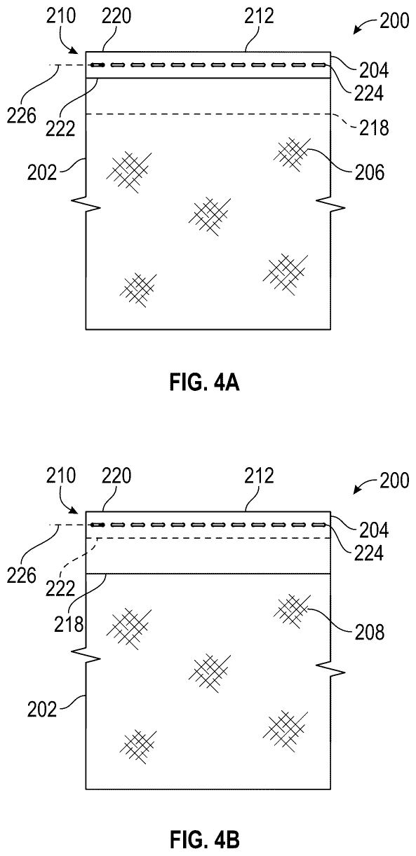

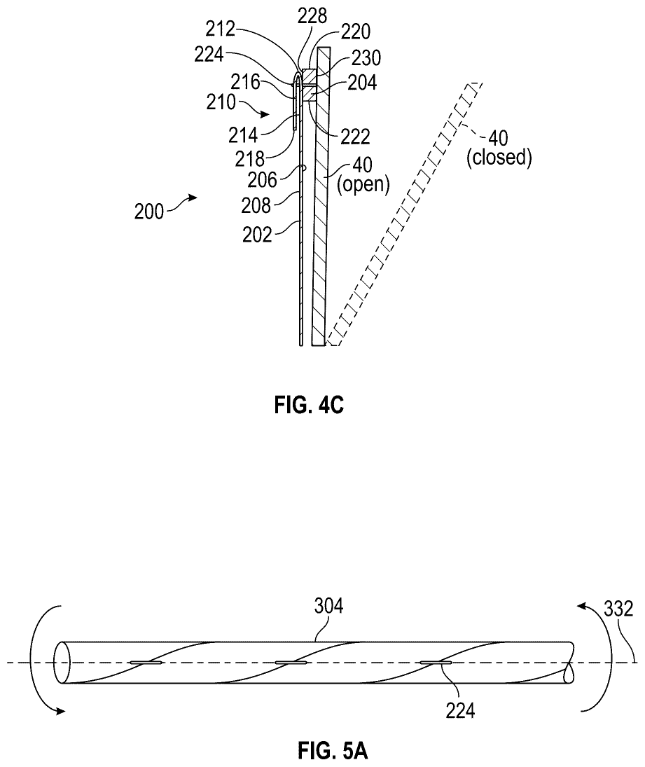

The protective member 60 is less abrasive to the leaflets 40 than the outer skirt 18 and is configured to shield a terminal end 58 of the outer skirt 18 from contacting the leaflets 40, especially when the leaflets 40 are in the open configuration. In some examples, the outflow end portion 50 of the outer skirt 18 can be folded so that the terminal end 58 is located below an outflow edge 54 of the outer skirt 18. In other examples, no fold is formed on the outflow end portion 50, and the outflow edge 54 is the terminal end 58 of the outer skirt 18. In some cases, the terminal end 58 (which can define the outflow edge 54 or located below the outflow edge 54) of the outer skirt 18 is cut. In some instances, the outer skirt is cut via heat and/or pre-treated by heat. The heat process(es) can cause the fabric to melt and then cool, which can form a relatively hard edge. Thus, by shielding the terminal end 58 of the outer skirt 18 from contacting the leaflets 40, the protective member 60 can, for example, not only reduce the wear and tear to the leaflets 40 but can also protect the edge of the outer skirt 18. Example skirt assemblies with protective members are described more fully below with references to FIGS. 4-7.

In some examples, as depicted in FIG. 2, the frame 12 can further include rungs of support members 64 extending between the axial frame members 28. The outflow end portion 50 of the outer skirt 18 can be further attached to the support members 64. The support members 64 can provide additional structural support of the outer skirt 18, for example, to reduce the likelihood that the outflow end portion 50 of the outer skirt 18 may extend through or loosely dangle across the outflow cells 30 (for example, due to the relatively large size of the outflow cells 30). Additional examples of frames having rungs of support members are described in U.S. Patent Application No. 63/343,986, which is incorporated by reference herein.

In certain examples, the prosthetic valve 10 can further include an inner skirt (not shown) arranged on and/or coupled to an inner surface of the frame 12. The inner skirt can be formed from any of various suitable biocompatible materials, including any of various synthetic materials, including fabrics (for example, polyethylene terephthalate fabric) or natural tissue (for example, pericardial tissue). The inner skirt can function as a sealing member to prevent or decrease perivalvular leakage, to anchor the leaflets 40 to the frame 12, and/or to protect the leaflets 40 against damage caused by contact with the frame 12 during crimping and during working cycles of the prosthetic valve 10. In some examples, the inflow edge of the leaflets 40 can be sutured to the inner skirt generally along the scallop line. The inner skirt can in turn be sutured to adjacent angled struts 24 of the frame 12. In other examples, the leaflets 40 can be sutured directly to the frame 12 along the scallop line via stitches.

Further details regarding the prosthetic valves and various components of prosthetic valves, including the related delivery apparatus/catheters/systems are described in WIPO Patent Application Publication No. WO 2018/222799, which is incorporated herein by reference.

### Exemplary Skirt Assembly Having Protective Members

As described above, the outer skirt can cover at least a lower portion of the outflow cells of the annular frame, and a cut edge of the outer skirt can extend between adjacent axial frame members. As described herein, a nonabrasive or less abrasive (compared to the outer skirt) protective member can be affixed to the outer skirt to shield the cut edge from the leaflets, thereby reducing wear and tear to the leaflets and/or protecting the cut of the outer skirt.

FIGS. 4A-4C and FIG. 6 depict examples of an outer skirt assembly 200 which can be incorporated in any of the prosthetic valves (for example, 10) described herein. For example, the outer skirt 18 of FIG. 1 can be part of the outer skirt assembly 200 that is disposed on an outer surface of the annular frame 12.

The outer skirt assembly 200 can include an outer skirt 202 and a protective member 204. The outer skirt 202 has an inner surface 206 and an outer surface 208. The protective member 204 can be attached to the inner surface 206 of the outer skirt 202. Thus, when attached to an annular frame, the inner surface 206 of the outer skirt 202 and the protective member 204 can face an interior space of the annular frame.

The protective member 204 is configured to shield a terminal end 218 (which can be a cut where yarns are fused together by thermal heating or other means) of the outer skirt 202 from contacting one or more leaflets 40 of the prosthetic valve. For example, as schematically depicted in FIG. 4C, when the leaflets 40 are in the open configuration, the leaflets 40 contact the protective member 204 instead of the terminal end 218 of the outer skirt 202. When the leaflets 40 are in the closed configuration, the leaflets 40 collapse inwardly and move away from the protective member 204. In FIG. 4C, the sizes of outer skirt 202, protective member 204, and leaflet 40 are not drawn to scale to better illustrate the protective member 204.

As depicted in FIG. 6, when attached to an annular frame 12, the protective member 204 can extend circumferentially around at least a portion of the annular frame 12 (for clarity, the leaflets are not shown in FIG. 6). In some examples, the protective member 204 can be a band or ribbon having a ring shape and encircle the full circumference of the annular frame 12. In other examples, the protective member 204 can comprise a plurality of separate pieces. For example, in some instances, the protective member can comprise 2, 3, 4, 5, 6, 7, 8, 9, 10, 11, 12, 13, 14, or 15 pieces. In particular examples, the protective member can comprise a separate piece extending between each adjacent pair of axial frame members 28 of the frame 12.

The protective member 204 is less abrasive than the outer skirt 202. The abrasiveness of a material can be measured by a coefficient of friction of the material. The larger the coefficient of friction, the more abrasive the material is. As described herein, the protective member 204 has a coefficient of friction less than 0.15. For example, the coefficient of friction of the protective member 204 can be between 0.1 and 1.0, or more specifically between 0.4 and 0.6. The protective member 204 is also configured to have a sufficiently high abrasion resistance so that the protective member 204 can withstand cyclic contact with the leaflets 40 during a lifetime of the prosthetic valve 10.

In certain examples, the outer skirt 202 can comprise a fabric material such as polyethylene terephthalate (PET). The fabric material of the outer skirt can, for example, be configured to reduce PVL and/or to help reduce migration of the prosthetic valve relative to the native tissue. In one particular example, the outer skirt 202 can include a first set of yarns and a second set of yarns that are woven together. The first set of yarns can be oriented at 45-degree angles relative to a longitudinal axis of the frame. The second set of yarns can be perpendicular to the first set of yarns. As described herein, the yarns can comprise multi-filament yarns (yarns comprising plural fibers or filaments) or mono-filament yarns (yarns comprising single fibers or filaments). The diameter of each filament can be between 5 μm and 20 μm (for example, about 10 μm). In other examples, the outer skirt 202 can include natural tissue (for example, pericardial tissue).

In some examples, the protective member 204 can include polytetrafluoroethylene (PTFE). As mentioned above, the material of the protective member can be selected to protect the prosthetic leaflets. For example, the protective member 204 can be a thick monofilament PTFE suture (for example, size 2-0, 3-0, etc.). In some examples, the protective member 204 can include thermoplastic polyurethane (TPU). In some examples, the protective member 204 can include microfilaments. As described herein, each microfilament has a diameter less than 1 μm. In certain examples, the material forming the microfilaments can be PET.

In some examples, the protective member 204 can be attached to an outflow end portion 210 of the outer skirt 202. The outer skirt 202 has an outflow edge 212. When attached to a frame, the outflow edge 212 defines an upper boundary of the outer skirt 202. The outflow edge 212 can be at different height relative to the frame 12. For example, the outflow edge 212 can be closer to or farther away from the outflow end 19 than the example depicted in FIG. 6. The protective member 204 has an outflow end 220 and an inflow end 222, which respectively define an upper boundary and a lower boundary of the protective member 204. As described herein, an “upper” part of a component of the outer skirt assembly 200 is located closer to an outflow end of a frame than a “lower” part of component when the outer skirt assembly 200 is attached to the frame.

In some examples, the outflow edge 212 can be a fold formed by folding the outflow end portion 210 of the outer skirt 202. As depicted in FIGS. 4A-4C, the outflow end portion 210 can be folded around the outflow edge 212 to form a double layer including a first layer portion 214 and a second layer portion 216. The terminal end 218 of the outer skirt 202 is located on the second layer portion 216. In the depicted example, the outflow end portion 210 is folded outwardly so that the first layer portion 214 (which can also be referred to as an “inner layer portion”) is positioned between the second layer portion 216 (which can also be referred to as an “outer layer portion”) and the protective member 204. Thus, in this configuration, the outer layer portion has the terminal end 218. In the depicted example, the terminal end 218 of the outer skirt 202 is located below the inflow end 222 of the protective member 204. In other examples, the terminal end 218 of the outer skirt 202 can be located between the outflow end 220 and the inflow end 222 of the protective member 204. In any case, the terminal end 218 of the outer skirt 202 can be shielded from the leaflets 40 by the protective member 204.

In another example, the outflow end portion 210 can be folded inwardly around the outflow edge 212 to form a double layer including the first layer portion 214 and the second layer portion 216. Similarly, the terminal end 218 of the outer skirt 202 is located on the second layer portion 216. In this configuration, the second layer portion 216 is positioned between the first layer portion 214 and the protective member 204. Thus, the first layer portion 214 becomes the outer layer portion and the second layer portion 216 becomes the inner layer portion. In this configuration, the inner layer portion has the terminal end 218. Further, the terminal end 218 of the outer skirt 202 can be located between the outflow end 220 and the inflow end 222 of the protective member 204. Thus, the terminal end 218 of the outer skirt 202 can still be shielded from the leaflets 40 by the protective member 204.

In the example depicted in FIGS. 4A-4C, the outflow end 220 of the protective member 204 is aligned with the outflow edge 212 of the outer skirt 202. Thus, when attached to a frame, the axial distance between an outflow end of the frame and the outflow end 220 of the protective member 204 is about the same as the axial distance between the outflow end of the frame and the outflow edge 212 of the outer skirt 202.

In another example, the outflow end 220 of the protective member 204 can be positioned above the outflow edge 212 of the outer skirt 202. Thus, when attached to a frame, the outflow end 220 of the protective member 204 is closer to an outflow end of the frame than the outflow edge 212 of the outer skirt 202. In such circumstances, the fold in the outflow end portion 210 of the outer skirt 202 can be optional. For example, the outflow edge 212 can be the terminal end 218 of the outer skirt 202. Because the protective member 202 extends above the outflow edge 212, the protective member 202 can still prevent the outflow edge 212 from contacting the leaflets 40.

In another example, the outflow end 220 of the protective member 204 can be positioned below the outflow edge 212 of the outer skirt 202. Thus, when attached to a frame, the outflow edge 212 of the outer skirt 202 is closer to an outflow end of the frame than the outflow end 220 of the protective member 204. In such scenarios, the outflow edge 212 can be a fold formed in the outflow end portion 210 of the outer skirt 202, as described above. Because of the fold, even if the leaflets 40 may contact the outflow edge 212, the terminal end 218 of the outer skirt 202 can still be shielded from the leaflets 40 by the protective member 202.

In yet another example, the protective member 204 can fold around the outflow edge 212 of the outer skirt 202. For example, the protective member 204 can be folded outwardly to form a pocket, and the outflow edge 212 of the outer skirt 202 can be received in the pocket. In such scenarios, the outflow edge 212 can be the terminal end 218 of the outer skirt 202. Thus, the pocket formed by the protective member 204 can prevent the terminal end 218 of the outer skirt 202 from contacting the leaflets 40.

In certain examples, the protective member 204 can be stitched to the outer skirt 202 via one or more sutures 224. When the outflow end portion 210 of the outer skirt 202 forms a fold, the sutures 224 can extend through both the first layer portion 214 and the second layer portion 216, as depicted in FIG. 4C.

In some examples, the one or more sutures 224 can form a stitch line 226 that is about parallel to the outflow edge 212 of the outer skirt 202. In some examples, the stitch line 226 and the outflow edge 212 can be separated by between 1 and 5 yarns of the outer skirt 202. In some examples, the stitch line 226 can be spaced apart from the outflow edge 212 by a distance ranging between 0.1 mm and 0.5 mm, or between 0.2 mm and 0.3 mm.

In some examples, the sutures 224 are concealed when viewed from an interior of the frame. For example, as depicted in FIG. 4C, the protective member 204 can have an outer surface 228 contacting the outer skirt 202 and an inner surface 230 facing the leaflets 40. The sutures 224 can extend through the outer surface 228 but do not extend out of the inner surface 230. As a result, the sutures 224 can also be shielded from the leaflets 40 by the protective member 204. In the depicted example, both the inner surface 230 and the outer surface 228 are flat or substantially flat. Note that in FIG. 4C, the thickness of the protective member 204 (measured between the inner surface 230 and the outer surface 228) is exaggerated to depict the suture 224 embedded therein. In practice, the thickness of the protective member 204 can be about the same as or even small then a thickness of the outer skirt 202.

In some examples, as depicted in FIGS. 4A-4B, the sutures 224 can extend out of the inner surface 230 of the protective member 204 and get exposed to the leaflets 40. In such cases, the sutures 224, like the protective member 204, can be configured to be less abrasive than the outer skirt 202. For example, the sutures 224 can comprise a less abrasive material with a lower coefficient of friction (than the fabric), such as PTFE, TPU, etc. As another example, the sutures 224 can comprise microfilaments with a diameter less than 1 μm. In one example, the material forming the microfilaments can be PET.

In addition to and/or in lieu of the sutures 224, the protective member 204 can be connected to the outer skirt 202 by other means. For example, the protective member 204 can be glued to the outflow end portion 210 of the outer skirt 202. In another example, the protective member 204 can be thermal fused with the outflow end portion 210 of the outer skirt 202.

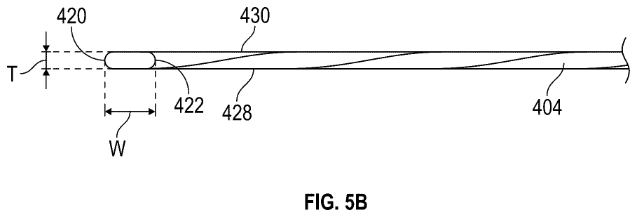

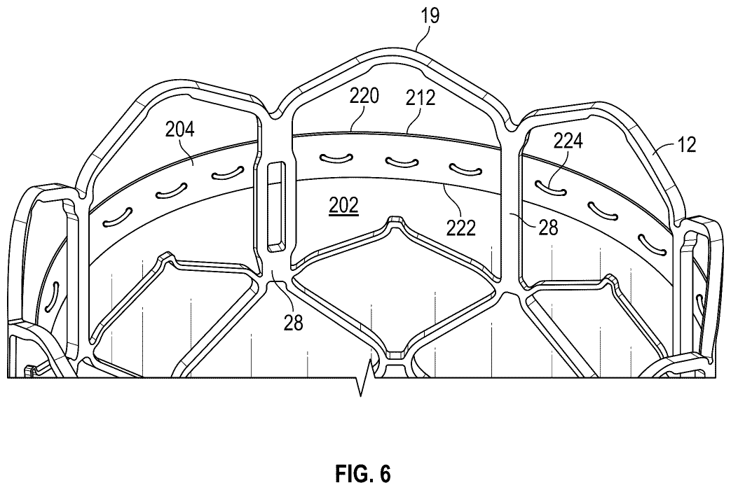

FIG. 5A shows a straightened protective member 304, according to one example. In certain cases, the protective member 304 can include a plurality of filaments extending along a longitudinal axis 332 of the protective member 304. If the filaments are all parallel to the longitudinal axis 332, the sutures 224, which form the stitch line 226, may extend between, but do not catch, some of the filaments.

In some examples, the protective member 304 can be twisted along the longitudinal axis 332 (as indicated by the two arrows). The degree of twisting can be predefined. In certain examples, the protective member 304 can be twisted between 5 and 50 rotations per inch. Specifically, the protective member 304 can be twisted between 10 and 14 rotations per inch.

Twisting of the protective member 304 can facilitate secure attachment of protective member 304 to the outer skirt 202 by the sutures 224. For example, by twisting the protective member 304, filaments within the protective member 304 are also twisted along the longitudinal axis 332, thus allowing the sutures 224 (which also extend along the longitudinal axis 332) to weave through and catch at least some of the filaments.

FIG. 5B shows a straightened protective member 404, according to another example. Similar to 304, the protective member 404 can be twisted along its longitudinal axis. In addition, the twisted protective member 404 can be compressed at an elevated temperature to shape set the protective member 404 to a flat configuration. For example, the protective member 404 can be shape set to have a flat inner surface 430 and a flat outer surface 428. The thickness (T) of the protective member 404 can be measured between the inner surface 430 and the outer surface 428. The width (W) of the protective member 404 can be measured between an outflow end 420 and an inflow end 422 of the protective member 404. In the flat configuration, the protective member 404 has a smaller thickness than the width. For example, the thickness of the protective member 404 can be less than 80%, 70%, 60%, 50%, 40%, 30%, 20%, or 10% of the width of the protective member 404. Reducing the thickness of the protective member 404 can reduce the impact of the protective member 404 on the crimped profile (for example, the smallest diameter) of the prosthetic valve.

In the examples described above, the outer skirt assembly 200 is described to include the outer skirt 202 and the protective member 204 (or 304, 404) is attached to the outer skirt 202. In other examples, the protective member 204 (or 304, 404) can be deemed a part of the outer skirt 202. For example, the outer skirt 202 can be considered to have a body portion corresponding to parts of outer skirt that are below the protective member 204 (or 304, 404) and an outflow end portion where the protective member 204 (or 304, 404) is located. The outflow end portion can have a fabric portion and the protective member 204 (or 304, 404) can be attached to the fabric portion, for example, by stitches, adhesive, or other means for attaching. Thus, the outflow end portion is less abrasive than the body portion. Similarly, the protective member 204 (or 304, 404) can shield a terminal end of the fabric portion from the leaflets of the prosthetic valve.

In some examples, the protective member can be an integral part of the outer skirt. For example, non-abrasive or less abrasive materials with low friction coefficients such as PTFE, TPU, etc., can be coated to the outflow end portion of the outer skirt to form a protective film that is fused with filaments of the outer skirt. The protective film can cover an outflow end portion of the outer skirt and prevent the leaflets to contact the terminal end of the outer skirt.

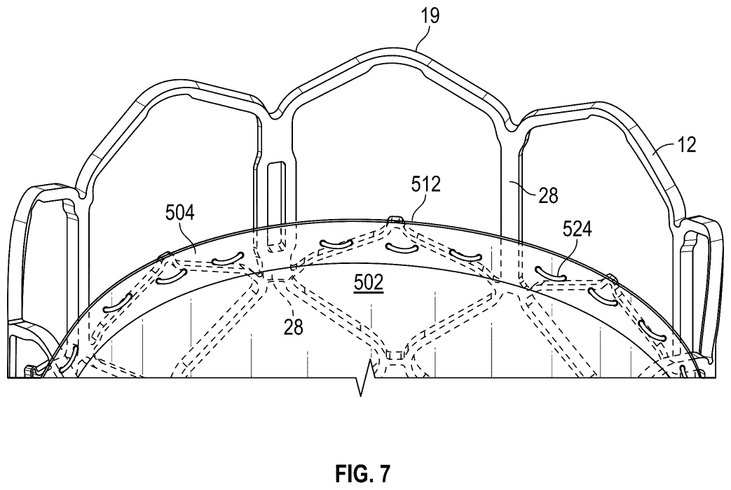

In some examples, a protective member similar to 204, 304, or 404 can also be added to an inner skirt of the prosthetic valve so as to form an inner skirt assembly. For example, FIG. 7 depicts a portion of the prosthetic valve 10, which shows an inner skirt 502 attached to an inner surface of the annular frame 12. For clarity, the outer skirt 202 disposed on the outer surface of the frame 12 and the leaflets are not shown in FIG. 7. As shown, a protective member 504, which is less abrasive than the inner skirt 502, can be attached to an inner surface of the inner skirt 502 (for examples, via sutures 524 that are similar to 224, or by other means). Thus, when the inner skirt 502 is mounted to the frame 12, the protective member 504 faces an interior space of the annular frame 12 (and leaflets of the prosthetic valve).

In some examples, the protective member 504 can be substantially the same as the protective member 204 described above in reference to FIGS. 4A-4C (or 304 of FIG. 5A, 404 of FIG. 5B). In some examples, the protective member 504 can be attached to the inner skirt 502 in substantially the same way as attaching the protective member 204 to the outer skirt 202 (for example, the protective member 204 and outer skirt 202 in FIGS. 4A-4C can be replaced by protective member 504 and inner skirt 502, respectively).

Similar to the outer skirt 202, the inner skirt 502 has an outflow edge 512 which can be a fold formed by folding an outflow end portion of the inner skirt (thus forming a double layer) or a terminal end of the inner skirt. The outflow edge 512 can be at different height relative to the frame 12. For example, the outflow edge 512 can be closer to or farther away from the outflow end 19 than the example depicted in FIG. 7. Similar to the protective member 204 (or 304, 404) which can shield the terminal end of the outer skirt 202 from contacting leaflets of the prosthetic valve, the protective member 504 can also be configured to shield the terminal end of the inner skirt 502 from contacting leaflets of the prosthetic valve. Thus, the protective member 504 can, for example, reduce the wear and tear to the leaflets and/or protect the terminal end of the inner skirt 502.

To assemble a prosthetic valve, a skirt member (for example, the outer skirt 202 or the inner skirt 502) having an annular shape can be prepared. Then a protective member (for example, 204, 304, 404, 504), which is less abrasive than the skirt member, can be affixed to an outflow end portion of the skirt member and encircles an inner surface of the skirt member. The protective member can be affixed to the skirt member by stitching, adhesive, thermal bonding, or other means for affixing. In some examples, before affixing the protective member to the skirt member, the protective member can be twisted along its length. In some examples, the twisted protective member can be compressed at a predefined temperature to shape set the protective member to have a flat configuration, as described above. In some examples, before affixing the protective member, the outflow end portion of the skirt member can be folded (either outwardly or inwardly) to create a double layer so that a terminal end of the skirt member is located below an outflow edge of the skirt member. After affixing the protective member to the skirt member, the skirt member can be attached to an annular frame of the prosthetic valve. For example, if the skirt member is an outer skirt, the skirt member can be mounted to an outer surface of the frame (e.g., via sutures or other means). If the skirt member is an inner skirt, the skirt member can be mounted to an inner surface of the frame (e.g., via sutures or other means).

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Exemplary Delivery Apparatus

FIG. 3 shows a delivery apparatus 100, according to an example, that can be used to implant an expandable prosthetic valve (for example, the prosthetic valve 10 and/or any of the other prosthetic valves described herein). In some examples, the delivery apparatus 100 can be specifically adapted for use in introducing a prosthetic valve into a heart.

The delivery apparatus 100 in the illustrated example of FIG. 3 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (for example, move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122, or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic valve in a radially compressed state. For example, as shown schematically in FIG. 3, a prosthetic device, such as the prosthetic valve 10, can be mounted around the balloon 118, at the valve mounting portion 124 of the delivery apparatus 100.

The balloon shoulder assembly, including the distal shoulder 126, can be configured to maintain the prosthetic valve 10 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 10 is mounted in the radially compressed state on the valve mounting portion 124 and during delivery of the prosthetic valve to the target implantation site. As such, the distal tip portion 128 can be configured to resist movement of the prosthetic valve 10 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 308 and an inner surface of the balloon 118. As such, fluid from the fluid source can flow to the fluid passageway from the annular space to inflate the balloon 118 and radially expand and deploy the prosthetic valve 10.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn can be operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 can be configured to adjust the axial position of the intermediate shaft 106 relative to the outer shaft 104 (for example, for fine positioning at the implantation site). Further details on the delivery apparatus 100 can be found in PCT Application No. PCT/US2021/047056, which is incorporated by reference herein.

Although the delivery apparatus 100 depicted in FIG. 3 is specifically adapted to deliver a balloon expandable prosthetic valve, it is to be understood that variants of the delivery apparatus 100 can be adapted for delivery of self-expandable prosthetic valves and/or mechanically expandable prosthetic valves, as described in references incorporated above.

### Exemplary Delivery Techniques

For implanting a prosthetic valve within the native aortic valve via a transfemoral delivery approach, the prosthetic valve can be mounted in a radially compressed state along the distal end portion of a delivery apparatus. The prosthetic valve and the distal end portion of the delivery apparatus can be inserted into a femoral artery and then advanced into and through the descending aorta, around the aortic arch, and through the ascending aorta. The prosthetic valve can be positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) can be introduced into the left ventricle through a surgical opening in the chest and the apex of the heart and the prosthetic valve can be positioned within the native aortic valve. Alternatively, in a transaortic procedure, a prosthetic valve (on the distal end portion of the delivery apparatus) can be introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve can be mounted in a radially compressed state along the distal end portion of a delivery apparatus. The prosthetic valve and the distal end portion of the delivery apparatus can be inserted into a femoral vein and then advanced into and through the inferior vena cava, into the right atrium, across the atrial septum (for example, through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) can be introduced into the left ventricle through a surgical opening in the chest and the apex of the heart and the prosthetic valve can be positioned within the native mitral valve.

For implanting a prosthetic valve within the native tricuspid valve, the prosthetic valve can be mounted in a radially compressed state along the distal end portion of a delivery apparatus. The prosthetic valve and the distal end portion of the delivery apparatus can be inserted into a femoral vein and then advanced into and through the inferior vena cava, and into the right atrium, and the prosthetic valve can be positioned within the native tricuspid valve. A similar approach can be used for implanting the prosthetic valve within the native pulmonary valve or the pulmonary artery, except that the prosthetic valve can be advanced through the native tricuspid valve into the right ventricle and toward the pulmonary valve/pulmonary artery.

Another delivery approach is a trans-atrial approach whereby a prosthetic valve (on the distal end portion of the delivery apparatus) can be inserted through an incision in the chest and an incision made through an atrial wall (of the right or left atrium) for accessing any of the native heart valves. Atrial delivery can also be made intravascularly, such as from a pulmonary vein. Still another delivery approach is a trans-ventricular approach whereby a prosthetic valve (on the distal end portion of the delivery apparatus) can be inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Exemplary Expansion Mechanisms of Prosthetic Valves

For any of the prosthetic valves described herein, the frame can be made of any of various suitable plastically-expandable materials. When constructed of a plastically-expandable material, the frame (and thus the prosthetic valve) can be crimped to a radially collapsed configuration on a delivery catheter or apparatus and then expanded inside a patient by an inflatable balloon or equivalent expansion mechanism. In certain examples, during delivery to the implantation site, the prosthetic valve can be placed inside of a delivery capsule or sheath to protect against the prosthetic valve contacting the patient's vasculature, such as when the prosthetic valve is advanced through a femoral artery. The capsule can also retain the prosthetic valve in a radially compressed state having a slightly smaller diameter and crimp profile than may be otherwise possible without a capsule by preventing any recoil (expansion) of the frame once it is crimped onto the delivery apparatus.

Suitable plastically-expandable materials that can be used to form the frame include, without limitation, stainless steel, a biocompatible, high-strength alloys (for example, a cobalt-chromium or a nickel-cobalt-chromium alloys), polymers, or combinations thereof. In particular examples, frame is made of a nickel-cobalt-chromium-molybdenum alloy, such as MP35N® alloy (SPS Technologies, Jenkintown, Pennsylvania), which is equivalent to UNS R30035 alloy (covered by ASTM F562-02). MP35N® alloy/UNS R30035 alloy comprises 35% nickel, 35% cobalt, 20% chromium, and 10% molybdenum, by weight.

Additional details on balloon expandable prosthetic valves can be found in U.S. Pat. No. 9,393,110, and U.S. Provisional Application No. 63/178,416, filed Apr. 22, 2021, U.S. Provisional Application No. 63/194,830, filed May 28, 2021, and U.S. Provisional Application No. 63/279,96, filed Nov. 13, 2021, all of which are incorporated by reference herein.

Any of the prosthetic valves described herein can be self-expandable. For example, the frame of the prosthetic valve can comprise a shape-memory material (for example, Nitinol). When the prosthetic valve is self-expandable, the frame (and thus the prosthetic valve) can be crimped to a radially compressed configuration and restrained in the compressed configuration by insertion into a sheath or equivalent mechanism of a delivery catheter. Once inside the body at the desired implantation site, the prosthetic valve can be deployed or released from the delivery sheath, which allows the prosthetic valve to expand to its functional size. In some examples, the frame (and therefore the prosthetic valve) can partially self-expand from the radially compressed configuration to a partially radially expanded configuration. The frame (and therefore the prosthetic valve) can be further radially expanded from the partially expanded configuration to a further radially expanded configuration via one or more actuation assemblies (for example, an inflatable balloon and/or one or more mechanical actuators) of the delivery apparatus.

Additional details regarding exemplary self-expandable prosthetic valves and the related delivery apparatus/catheters/systems are described in U.S. Pat. Nos. 8,652,202, 9,155,619, and 9,867,700, all of which are incorporated herein by reference.

Additionally, and/or alternatively, any of the prosthetic valves described herein can be mechanically expandable. For example, the struts of the frame can be pivotably coupled to one another at one or more pivot joints along the length of each strut. As a result, an axial force applied to the frame (for example, pressing the inflow end and the outflow end of the frame toward each other or pulling the inflow end and the outflow end of the frame away from each other) can cause the prosthetic valve to radially expand or compress. The axial force can be generated by actuating one or more mechanical actuators of the delivery apparatus that are operatively coupled to the frame.

Additional details regarding exemplary mechanically-expandable prosthetic valves and the related delivery apparatus/catheters/systems are described in U.S. Patent Application Publication Nos. 2018/0153689, 2018/0311039, 2019/0060057, and PCT Patent Application Publication No. WO/2021/188476, all of which are incorporated by reference herein.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve comprising: an annular frame that is radially expandable and compressible; a skirt member attached to the annular frame; and a protective member attached to an inner surface the skirt member, wherein the protective member is less abrasive to leaflets of the prosthetic valve than the skirt member and extends circumferentially around at least a portion of the annular frame.

Example 2. The prosthetic valve of claim 1, wherein the skirt member is an outer skirt disposed on an outer surface of the annular frame.

Example 3. The prosthetic valve of any example herein, particularly example 1, wherein the skirt member is an inner skirt disposed on an inner surface of the annular frame.

Example 4. The prosthetic valve of any example herein, particularly any one of examples 1-3, wherein the protective member comprises polytetrafluoroethylene (PTFE), wherein the skirt member comprises polyethylene terephthalate (PET).

Example 5. The prosthetic valve of any example herein, particularly any one of examples 1-4, wherein the protective member is attached to an outflow end portion of the skirt member.

Example 6. The prosthetic valve of any example herein, particularly example 5, wherein an outflow end of the protective member is aligned with an outflow edge of the skirt member.

Example 7. The prosthetic valve of any example herein, particularly example 5, wherein an outflow end of the protective member is closer to an outflow end of the frame than an outflow edge of the skirt member.

Example 8. The prosthetic valve of any example herein, particularly example 5, wherein an outflow edge of the skirt member is closer to an outflow end of the frame than an outflow end of the protective member.

Example 9. The prosthetic valve of any example herein, particularly any one of examples 1-5, wherein the protective member folds around an outflow edge of the skirt member.

Example 10. The prosthetic valve of any example herein, particularly any one of examples 6-9, wherein the skirt member is folded around the outflow edge to form an inner layer portion and an outer layer portion, wherein the inner layer portion is positioned between the outer layer portion and the protective member.

Example 11. The prosthetic valve of any example herein, particularly example 10, wherein the outer layer portion comprises a terminal end of the skirt member.

Example 12. The prosthetic valve of any example herein, particularly example 10, wherein the inner layer portion comprises a terminal end of the skirt member.

Example 13. The prosthetic valve of any example herein, particularly any one of examples 1-12, wherein the protective member is stitched to the skirt member via one or more sutures.

Example 14. The prosthetic valve of any example herein, particularly example 13, wherein the one or more sutures are concealed when viewed from an interior of the frame.

Example 15. The prosthetic valve of any example herein, particularly any one of examples 1-14, wherein the protective member has a first flat surface contacting the skirt member and a second flat surface opposite to the first flat surface.

Example 16. The prosthetic valve of any example herein, particularly any one of examples 1-15, wherein the protective member is twisted along a longitudinal axis of the protective member.

Example 17. A prosthetic valve comprising: an annular frame that is radially expandable and compressible; and a skirt assembly attached to the annular frame, wherein the skirt assembly comprises a skirt member and a protective member attached to an inner surface of the skirt member, wherein the protective member is located at an outflow end portion of the skirt member and extends circumferentially around at least a portion of the annular frame.

Example 18. The prosthetic valve of any example herein, particularly example 16, wherein the skirt member is an outer skirt disposed on an outer surface of the annular frame.

Example 19. The prosthetic valve of any example herein, particularly example 16, wherein the skirt member is an inner skirt disposed on an inner surface of the annular frame.

Example 20. The prosthetic valve of any example herein, particularly any one of examples 17-19, wherein the protective member is less abrasive to leaflets of the prosthetic valve than the skirt member.

Example 21. The prosthetic valve of any example herein, particularly any one of examples 17-20, wherein the protective member comprises polytetrafluoroethylene (PTFE).

Example 22. The prosthetic valve of any example herein, particularly any one of examples 17-21, wherein the protective member comprises thermoplastic polyurethane (TPU).

Example 23. The prosthetic valve of any example herein, particularly any one of examples 17-22, wherein the protective member comprises microfilaments with a diameter less than 1 μm.

Example 24. The prosthetic valve of any example herein, particularly any one of examples 17-23, wherein the protective member is stitched to the skirt member via one or more sutures.

Example 25. The prosthetic valve of any example herein, particularly example 24, wherein the one or more sutures comprise polytetrafluoroethylene (PTFE).

Example 26. The prosthetic valve of any example herein, particularly any one of examples 24-25, wherein the one or more sutures comprise microfilaments with a diameter less than 1 μm.

Example 27. The prosthetic valve of any example herein, particularly any one of examples 24-26, wherein the protective member has a first surface contacting the skirt member and a second surface opposite to the first surface, wherein the one or more sutures do not extend out of the second surface.

Example 28. The prosthetic valve of any example herein, particularly any one of examples 24-27, wherein the skirt member comprises a fold that defines an outflow edge of the skirt member, wherein the fold is formed by an inner layer portion and an outer layer portion, wherein the inner layer portion is positioned between the outer layer portion and the protective member.

Example 29. The prosthetic valve of any example herein, particularly example 28, wherein the one or more sutures are stitched to both the inner layer portion and the outer layer portion.

Example 30. The prosthetic valve of any example herein, particularly any one of examples 28-29, wherein the outer layer portion comprises a terminal end of the skirt member.

Example 31. The prosthetic valve of any example herein, particularly any one of examples 28-29, wherein the inner layer portion comprises a terminal end of the skirt member, wherein the terminal end is shielded by the protective member.

Example 32. The prosthetic valve of any example herein, particularly any one of examples 17-31, wherein the protective member is twisted along a longitudinal axis of the protective member.

Example 33. The prosthetic valve of any example herein, particularly any one of examples 17-32, wherein the protective member has flat inner and outer surfaces.

Example 34. The prosthetic valve of any example herein, particularly any one of examples 17-33, wherein the skirt member comprises polyethylene terephthalate (PET).

Example 35. A prosthetic valve comprising: an annular frame that is radially expandable and compressible; and a skirt member attached to the annular frame, wherein the skirt member comprises a body portion and an outflow end portion, wherein the outflow end portion is less abrasive than the body portion.

Example 36. The prosthetic valve of any example herein, particularly example 35, wherein the skirt member is an outer skirt disposed on an outer surface of the annular frame.

Example 37. The prosthetic valve of any example herein, particularly example 35, wherein the skirt member is an inner skirt disposed on an inner surface of the annular frame.

Example 38. The prosthetic valve of any example herein, particularly any one of examples 35-37, wherein the outflow end portion of the skirt member comprises a fabric portion and a protective member attached to the fabric portion, wherein the protective member extends circumferentially around at least a portion of the frame.

Example 39. The prosthetic valve of any example herein, particularly example 38, wherein the protective member comprises polytetrafluoroethylene (PTFE).

Example 40. The prosthetic valve of any example herein, particularly any one of examples 38-39, wherein the protective member comprises thermoplastic polyurethane (TPU).

Example 41. The prosthetic valve of any example herein, particularly any one of examples 38-40, wherein the protective member comprises microfilaments with a diameter less than 1 μm.

Example 42. The prosthetic valve of any example herein, particularly any one of examples 38-41, wherein the protective member is attached to an inner surface of the fabric portion.

Example 43. The prosthetic valve of any example herein, particularly any one of examples 38-42, wherein the protective member is stitched to the fabric portion via one or more sutures.

Example 44. The prosthetic valve of any example herein, particularly example 43, wherein the one or more sutures comprise polytetrafluoroethylene (PTFE).

Example 45. The prosthetic valve of any example herein, particularly any one of examples 43-44, wherein the one or more sutures comprise microfilaments with a diameter less than 1 μm.

Example 46. The prosthetic valve of any example herein, particularly any one of examples 43-45, wherein the protective member has a first surface contacting the fabric portion and a second surface opposite to the first surface, wherein the one or more sutures do not extend out of the second surface.

Example 47. The prosthetic valve of any example herein, particularly any one of examples 43-46, wherein the fabric portion is folded around an outflow edge of the skirt member to form an inner layer portion that contacts the protective member and an outer layer portion that is separated from the protective member by the inner layer portion.

Example 48. The prosthetic valve of any example herein, particularly example 47, wherein the one or more sutures are stitched to both the inner layer portion and the outer layer portion.

Example 49. The prosthetic valve of any example herein, particularly any one of examples 47-48, wherein the outer layer portion comprises a terminal end of the skirt member.

Example 50. The prosthetic valve of any example herein, particularly any one of examples 46-49, wherein the one or more sutures are spaced apart from the outflow edge of the skirt member by a distance ranging between 0.1 mm and 0.5 mm.

Example 51. The prosthetic valve of any example herein, particularly example 50, wherein the one or more sutures are spaced apart from the outflow edge of the skirt member by a distance ranging between 0.2 mm and 0.3 mm.

Example 52. The prosthetic valve of any example herein, particularly any one of examples 38-51, wherein the protective member is glued to the fabric portion.

Example 53. The prosthetic valve of any example herein, particularly any one of examples 38-52, wherein the protective member is twisted along a longitudinal axis the protective member.

Example 54. The prosthetic valve of any example herein, particularly example 53, wherein the protective member is twisted between 5 and 50 rotations per inch.

Example 55. The prosthetic valve of any example herein, particularly example 54, wherein the protective member is twisted between 10 and 14 rotations per inch.

Example 56. The prosthetic valve of any example herein, particularly any one of examples 35-55, wherein the protective member has flat inner and outer surfaces.

Example 57. The prosthetic valve of any example herein, particularly any one of examples 35-56, wherein the body portion comprises polyethylene terephthalate (PET).

Example 58. The prosthetic valve of any example herein, particularly any one of examples 35-57, wherein the body portion comprises a first set of yarns and a second set of yarns, wherein the first set of yarns are oriented at 45-degree angles relative to a longitudinal axis of the frame, wherein the second set of yarns are perpendicular to the first set of yarns.

Example 59. The prosthetic valve of any example herein, particularly any one of examples 35-58, wherein the annular frame comprises an inflow end, an outflow end, a first row of angled struts defining the outflow end, a second row of angled struts being closer to the inflow end than the first row of angled struts, and a plurality of axial frame members bridging the first row of angled struts and the second row of angled struts, wherein the outflow end portion of the skirt member is attached to the plurality of axial frame members.

Example 60. The prosthetic valve of any example herein, particularly example 59, further comprising a leaflet structure positioned within the annular frame and configured to permit blood flow from the inflow end to the outflow end and block blood fluid flow from the outflow end to the inflow end.

Example 61. The prosthetic valve of any example herein, particularly example 60, further comprising an inner skirt attached to an inner surface of the annular frame, wherein the leaflet structure is connected to the inner skirt.

Example 62. The prosthetic valve of any example herein, particularly any one of examples 59-61, wherein the first row of angled struts, the second row of angled struts, and the plurality of axial frame members define a plurality of outflow cells of the annular frame, wherein the skirt member covers at least a portion of the outflow cells.

Example 63. A prosthetic valve comprising: an annular frame that is radially expandable and compressible; a plurality of leaflets mounted inside the annular frame; a skirt member attached to the annular frame; and a protective member attached to an inner surface the skirt member, wherein the plurality of leaflets are movable between an open configuration and a closed configuration, wherein when in the open configuration, the plurality of leaflets contact the protective member.

Example 64. The prosthetic valve of any example herein, particularly example 63, wherein the skirt member is an outer skirt disposed on an outer surface of the annular frame.

Example 65. The prosthetic valve of any example herein, particularly example 63, wherein the skirt member is an inner skirt disposed on an inner surface of the annular frame.

Example 66. The prosthetic valve of any example herein, particularly any one of examples 63-65, wherein the protective member is less abrasive to the leaflets than the skirt member.

Example 67. The prosthetic valve of any example herein, particularly any one of examples 63-66, wherein the protective member extends circumferentially around at least a portion of the annular frame.

Example 68. The prosthetic valve of any example herein, particularly any one of examples 63-67, wherein the protective member comprises polytetrafluoroethylene (PTFE).

Example 69. The prosthetic valve of any example herein, particularly any one of examples 63-68, wherein the skirt member comprises polyethylene terephthalate (PET).

Example 70. The prosthetic valve of any example herein, particularly any one of examples 63-69, wherein the protective member shields a terminal end of the skirt member from the plurality of leaflets when the plurality of leaflets are in the open configuration.

Example 71. The prosthetic valve of any example herein, particularly example 70, wherein the skirt member comprises a fold formed by an inner layer portion and an outer layer portion, wherein the fold defines an outflow edge of the skirt member.

Example 72. The prosthetic valve of any example herein, particularly example 71, wherein the outer layer portion comprises the terminal end of the skirt member.

Example 73. The prosthetic valve of any example herein, particularly example 71, wherein the inner layer portion comprises the terminal end of the skirt member.

Example 74. The prosthetic valve of any example herein, particularly any one of examples 63-73, wherein the protective member is stitched to the skirt member via one or more sutures.

Example 75. The prosthetic valve of any example herein, particularly example 74, wherein the one or more sutures comprise polytetrafluoroethylene (PTFE).

Example 76. The prosthetic valve of any example herein, particularly any one of examples 74-75, wherein the one or more sutures do not extend out of an inner surface of the protective member.

Example 77. The prosthetic valve of any example herein, particularly any one of examples 63-76, wherein a thickness of the protective member measured along a radial axis of the frame is smaller than a width of the protective member measured along an axial axis of the frame.

Example 78. The prosthetic valve of any example herein, particularly any one of examples 63-77, wherein the protective member is twisted along a longitudinal axis of the protective member.

Example 79. A prosthetic valve comprising: an annular frame that is radially expandable and compressible; a skirt member attached to the annular frame; and a protective member attached to an inner surface the skirt member, wherein the protective member is configured to shield a terminal end of the skirt member from contacting one or more leaflets mounted within the annular frame.

Example 80. The prosthetic valve of any example herein, particularly example 79, wherein the skirt member is an outer skirt disposed on an outer surface of the annular frame.

Example 81. The prosthetic valve of any example herein, particularly example 79, wherein the skirt member is an inner skirt disposed on an inner surface of the annular frame.

Example 82. The prosthetic valve of any example herein, particularly any one of examples 79-81, further comprising the one or more leaflets mounted within the annular frame, wherein the one or more leaflets are configured to permit a flow of blood from an inflow end to an outflow end of the prosthetic valve and block the flow of blood from the outflow end to the inflow end of the prosthetic valve.

Example 83. The prosthetic valve of any example herein, particularly any one of examples 79-82, wherein the protective member is less abrasive to the one or more leaflets than the skirt member.

Example 84. The prosthetic valve of any example herein, particularly any one of examples 79-83, wherein the protective member encircles the annular frame.

Example 85. The prosthetic valve of any example herein, particularly any one of examples 79-84, wherein the skirt member comprises a fold formed by an inner layer portion and an outer layer portion.

Example 86. The prosthetic valve of any example herein, particularly example 85, wherein the outer layer portion comprises the terminal end of the skirt member.

Example 87. The prosthetic valve of any example herein, particularly example 85, wherein the inner layer portion comprises the terminal end of the skirt member.

Example 88. The prosthetic valve of any example herein, particularly any one of examples 85-87, wherein the protective member is stitched to the skirt member via one or more sutures.

Example 89. The prosthetic valve of any example herein, particularly example 88, wherein the one or more sutures extend through both the inner layer portion and the outer layer portion.

Example 90. The prosthetic valve of any example herein, particularly any one of examples 79-89, wherein the protective member is twisted along a longitudinal axis of the protective member.

Example 91. A skirt assembly for a prosthetic valve, comprising: a skirt member having an annular shape; and a protective member attached to an outflow end portion of the skirt member, wherein the protective member extends circumferentially around an inner surface of the skirt member, wherein the protective member is less abrasive to leaflets of the prosthetic valve than the skirt member.

Example 92. The skirt assembly of any example herein, particularly example 91, wherein the skirt member is an outer skirt configured to be disposed on an outer surface of an annular frame of the prosthetic valve.

Example 93. The prosthetic valve of any example herein, particularly example 91, wherein the skirt member is an inner skirt configured to be disposed on an inner surface of an annular frame of the prosthetic valve.

Example 94. The skirt assembly of any example herein, particularly any one of examples 91-93, wherein the protective member comprises polytetrafluoroethylene (PTFE) or thermoplastic polyurethane (TPU).

Example 95. The skirt assembly of any example herein, particularly any one of examples 91-94, wherein the protective member comprises microfilaments with a diameter less than 1 μm.

Example 96. The prosthetic valve of any example herein, particularly any one of examples 91-95, wherein the protective member is stitched to the skirt member via one or more sutures.

Example 97. The skirt assembly of any example herein, particularly example 96, wherein the protective member is twisted along a longitudinal axis of the protective member.

Example 98. The skirt assembly of any example herein, particularly example 97, wherein the one or more sutures weave through twisted filaments of the protective member.

Example 99. The skirt assembly of any example herein, particularly any one of examples 96-98, wherein the protective member has a first surface contacting the outflow end portion of the skirt member and a second surface opposite to the first surface, wherein the one or more sutures do not extend out of the second surface.

Example 100. The skirt assembly of any example herein, particularly example 99, wherein the first surface and the second surface are flat.

Example 101. The skirt assembly of any example herein, particularly any one of examples 96-100, wherein the one or more sutures comprise polytetrafluoroethylene (PTFE).

Example 102. The skirt assembly of any example herein, particularly any one of examples 96-101, wherein the one or more sutures comprise microfilaments with a diameter less than 1 μm.

Example 103. The skirt assembly of any example herein, particularly any one of examples 91-102, wherein the skirt member comprises polyethylene terephthalate (PET).

Example 104. The skirt assembly of any example herein, particularly any one of examples 91-103, wherein the outflow end portion of the skirt member is folded outwardly so as to define a fold aligned with an outflow end of the protective member.

Example 105. An assembly comprising: a prosthetic valve of any example herein, particularly any one of examples 1-90; and a delivery apparatus configured to deliver the prosthetic valve in a radially compressed state to a target location.

Example 106. The assembly of any example herein, particularly example 105, wherein the delivery apparatus comprises a balloon shaft and a balloon mounted on a distal end portion of the balloon shaft, wherein the prosthetic valve is crimped on the balloon when delivering the prosthetic valve to the target location.

Example 107. The assembly of any example herein, particularly any one of examples 105-106, wherein the prosthetic valve is self-expandable from the radially compressed state to a radially expanded state, wherein the delivery apparatus comprises an outer sheath configured to retain the prosthetic valve in the radially compressed state when delivering the prosthetic valve to the target location.

Example 108. The assembly of any example herein, particularly any one of examples 105-107, wherein the delivery apparatus comprises an actuator coupled to an expansion mechanism of the prosthetic valve, wherein actuation of the actuator is configured to radially expand the prosthetic valve from the radially compressed state to the radially expanded state.

Example 109. A method of assembling a prosthetic valve, the method comprising: preparing a skirt member having an annular shape; and affixing a protective member to an outflow end portion of the skirt member, wherein the protective member encircles an inner surface of the skirt member, wherein the protective member is less abrasive to leaflets of the prosthetic valve than the skirt member.

Example 110. The method of any example herein, particularly example 109, wherein the affixing comprises stitching the protective member to the inner surface of the skirt member.

Example 111. The method of any example herein, particularly any one of examples 109-110, wherein the affixing comprises gluing the protective member to the inner surface of the skirt member.

Example 112. The method of any example herein, particularly any one of examples 109-111, wherein the affixing comprises thermal bonding the protective member to the inner surface of the skirt member.

Example 113. The method of any example herein, particularly any one of examples 109-112, further comprising twisting the protective member along a longitudinal axis of the protective member.

Example 114. The method of any example herein, particularly any one of examples 109-113, further comprising compressing the protective member at a predefined temperature to shape-set the protective member to have flat inner and outer surfaces.

Example 115. The method of any example herein, particularly any one of examples 109-110, further comprising folding the skirt member outwardly along an outflow end of the protective member.

Example 116. The method of any example herein, particularly any one of examples 109-115, further comprising attaching the skirt member to an outer surface of an annular frame of the prosthetic valve.

Example 117. The method of any example herein, particularly any one of examples 109-115, further comprising attaching the skirt member to an inner surface of an annular frame of the prosthetic valve.

Example 118. A method comprising: delivering a prosthetic device in a radially compressed state to a target location; and radially expanding the prosthetic device to a radially expanded state, wherein the prosthetic device is a prosthetic valve according to any example herein, particularly any one of examples 1-90.

Example 119. A method comprising sterilizing the prosthetic valve of any example herein, particularly any one of examples 1-90, sterilizing the skirt assembly of any example herein, particularly any one of examples 91-104, or sterilizing the assembly of any example herein, particularly any one of examples 105-108.

The features described herein with regard to any example can be combined with other features described in any one or more of the other examples, unless otherwise stated. For example, any one or more of the features of one prosthetic valve can be combined with any one or more features of another prosthetic valve.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A prosthetic valve comprising:
an annular frame that is radially expandable and compressible;
a skirt member attached to the annular frame; and
a protective member attached to an inner surface the skirt member,
wherein the protective member is less abrasive to leaflets of the prosthetic valve than the skirt member and extends circumferentially around at least a portion of the annular frame.

2. The prosthetic valve of claim 1, wherein the skirt member is an outer skirt disposed on an outer surface of the annular frame.

3. The prosthetic valve of claim 1, wherein the skirt member is an inner skirt disposed on an inner surface of the annular frame.

4. The prosthetic valve of claim 1, wherein the protective member comprises polytetrafluoroethylene (PTFE), wherein the skirt member comprises polyethylene terephthalate (PET).

5. The prosthetic valve of claim 1, wherein the protective member is attached to an outflow end portion of the skirt member.

6. The prosthetic valve of claim 5, wherein an outflow end of the protective member is aligned with an outflow edge of the skirt member.

7. The prosthetic valve of claim 5, wherein an outflow end of the protective member is closer to an outflow end of the frame than an outflow edge of the skirt member.

8. The prosthetic valve of claim 5, wherein an outflow edge of the skirt member is closer to an outflow end of the frame than an outflow end of the protective member.

9. The prosthetic valve of claim 1, wherein the protective member folds around an outflow edge of the skirt member.

10. The prosthetic valve of claim 6, wherein the skirt member is folded around the outflow edge to form an inner layer portion and an outer layer portion, wherein the inner layer portion is positioned between the outer layer portion and the protective member.

11. The prosthetic valve of claim 1, wherein the protective member is stitched to the skirt member via one or more sutures.

12. The prosthetic valve of claim 11, wherein the one or more sutures are concealed when viewed from an interior of the frame.

13. A prosthetic valve comprising:
an annular frame that is radially expandable and compressible; and
a skirt assembly attached to the annular frame,
wherein the skirt assembly comprises a skirt member and a protective member attached to an inner surface of the skirt member, wherein the protective member is located at an outflow end portion of the skirt member and extends circumferentially around at least a portion of the annular frame.

14. The prosthetic valve of claim 13, wherein the protective member is less abrasive to leaflets of the prosthetic valve than the skirt member.

15. The prosthetic valve of claim 13, wherein the protective member comprises microfilaments with a diameter less than 1 μm.

16. The prosthetic valve of claim 13, wherein the protective member is stitched to the skirt member via one or more sutures.

17. The prosthetic valve of claim 13, wherein the protective member is twisted along a longitudinal axis of the protective member.

18. The prosthetic valve of claim 13, wherein the protective member has flat inner and outer surfaces.

19. A prosthetic valve comprising:
an annular frame that is radially expandable and compressible; and
a skirt member attached to the annular frame, wherein the skirt member comprises a body portion and an outflow end portion, wherein the outflow end portion is less abrasive than the body portion.

20. The prosthetic valve of claim 19, wherein the outflow end portion of the skirt member comprises a fabric portion and a protective member attached to the fabric portion, wherein the protective member extends circumferentially around at least a portion of the frame.
